(19) United States
(12) Patent Application Publication
(10) Pub. No.: US 2019/0320992 A1
(43) Pub. Date: Oct. 24, 2019

(54) X-RAY DIAGNOSTIC APPARATUS, MEDICAL IMAGE PROCESSING APPARATUS, AND MEDICAL IMAGE DIAGNOSIS APPARATUS

(71) Applicant: Canon Medical Systems Corporation, Otawara-shi (JP)

(72) Inventors: Takashi KOYAKUMARU, Nasushiobara (JP); Nobuhide OOI, Nasushiobara (JP); Koji ANDO, Otawara (JP); Mitsuo AKIYAMA, Otawara (JP); Sayaka TAKAHASHI, Nasushiobara (JP); Ryoichi NAGAE, Nasushiobara (JP); Minori IZUMI, Shioya (JP); Takayuki KOJIMA, Utsunomiya (JP)

(73) Assignee: Canon Medical Systems Corporation, Otawara-shi (JP)

(21) Appl. No.: 16/382,827

(22) Filed: Apr. 12, 2019

(30) Foreign Application Priority Data
Apr. 20, 2018 (JP) 2018-081316

(51) Int. Cl. A61B 6/00; A61B 8/08; A61B 5/055

(52) U.S. Cl. CPC A61B 6/504; A61B 6/5247; A61B 8/485; A61B 6/463; A61B 8/0891; A61B 5/055; A61B 8/0858

(57) ABSTRACT

The X-ray diagnostic apparatus according to a present embodiment includes processing circuitry. The processing circuitry is configured to: generate an X-ray image of an object by irradiating the object with X-rays; acquire an evaluation result of at least one of elasticity evaluation of a blood vessel of the object and thickness evaluation of a wall of the blood vessel of the object; generate a superimposed image in which the evaluation result is superimposed on the X-ray image; and display the superimposed image on a display.

### CROSS-REFERENCE TO RELATED APPLICATION

This application is based upon and claims the benefit of priority from Japanese Patent Application No. 2018-081316, filed on Apr. 20, 2018, the entire contents of each of which are incorporated herein by reference.

### FIELD

An embodiment as an aspect of the present invention relates to an X-ray diagnostic apparatus, a medical image processing apparatus, and a medical image diagnosis apparatus.

### BACKGROUND

For the purpose of improving treatment efficiency by using different types of medical image diagnosis apparatuses in combination, there is known a medical image diagnosis system equipped with different types of medical image diagnosis apparatuses such as an X-ray diagnostic apparatus, an ultrasonic diagnostic apparatus, an X-ray CT (Computed Tomography) apparatus, and a magnetic resonance imaging apparatus. For instance, when an interventional treatment using a catheter is performed, the X-ray diagnostic apparatus and the ultrasonic diagnostic apparatus are used in combination.

The X-ray diagnostic apparatus is a diagnostic apparatus configured to transmit X-rays through an object and generate the object image by using a transmission image. As a means for acquiring X-ray images, there are “a radiographic mode” in which relatively strong X-rays are radiated and “a fluoroscopic mode” in which relatively weak X-rays are radiated. A doctor inserts the catheter into the patient while checking the catheter in the blood vessel by X-ray irradiation in the radiographic mode or fluoroscopic mode. After the catheter reaches the affected area, imaging of the affected area is performed from various angles by X-rays. Thereafter, the identified affected area is treated with the catheter. In order not to overlook a lesion that cannot be checked by fluoroscopy and/or radiography with the use of X-rays, there has been increasing interest on a method of identifying the affected area by using the ultrasonic diagnostic apparatus in combination.

In the X-ray fluoroscopy and X-ray radiography in combination with ultrasonic images, an abdominal-aorta stent-graft interpolation is performed, for instance. The abdominal-aorta stent-graft interpolation refers to a procedure of inserting a catheter with a stent graft (i.e., artificial blood vessel) attached to its tip into a blood vessel of a patient and then placing the stent graft in the blood vessel under the interventional radiology (IVR). The abdominal-aorta stent-graft interpolation is aimed at blocking blood flow to the aneurysm generated inside the aorta and preventing rupture of the aneurysm.

### BRIEF DESCRIPTION OF THE DRAWINGS

FIG. 1 is a schematic diagram illustrating a configuration of a medical image diagnosis system according to a first embodiment.

FIG. 2 is a diagram illustrating an appearance of the medical image diagnosis system according to the first embodiment.

FIG. 3 is a diagram as a flowchart illustrating an operation of the medical image diagnosis system according to the first embodiment.

FIG. 4 is a first example of a displayed superimposed image in the medical image diagnosis system according to the first embodiment.

FIG. 5 is a second example of a displayed superimposed image in the medical image diagnosis system according to the first embodiment.

FIG. 6 is a third example of a displayed superimposed image in the medical image diagnosis system according to the first embodiment.

FIG. 7 is a schematic diagram illustrating a configuration of a medical image diagnosis system according to a second embodiment.

FIG. 8 is a schematic diagram illustrating a configuration of a medical image diagnosis system according to a third embodiment.

### DETAILED DESCRIPTION

An X-ray diagnostic apparatus, a medical image processing apparatus, and a medical image diagnosis system according to a present embodiment will be described with reference to the accompanying drawings.

The X-ray diagnostic apparatus according to a present embodiment includes processing circuitry. The processing circuitry is configured to: generate an X-ray image of an object by irradiating the object with X-rays; acquire an evaluation result of at least one of elasticity evaluation of a blood vessel of the object and thickness evaluation of a wall of the blood vessel of the object; generate a superimposed image in which the evaluation result is superimposed on the X-ray image; and display the superimposed image on a display.

### First Embodiment

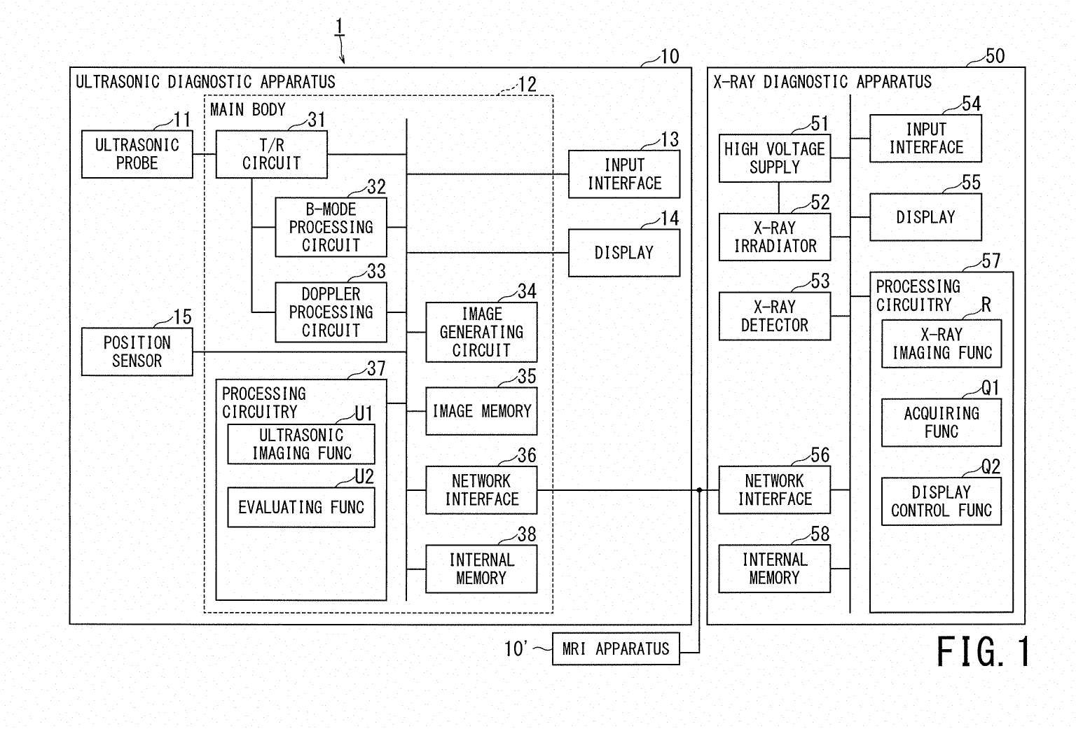

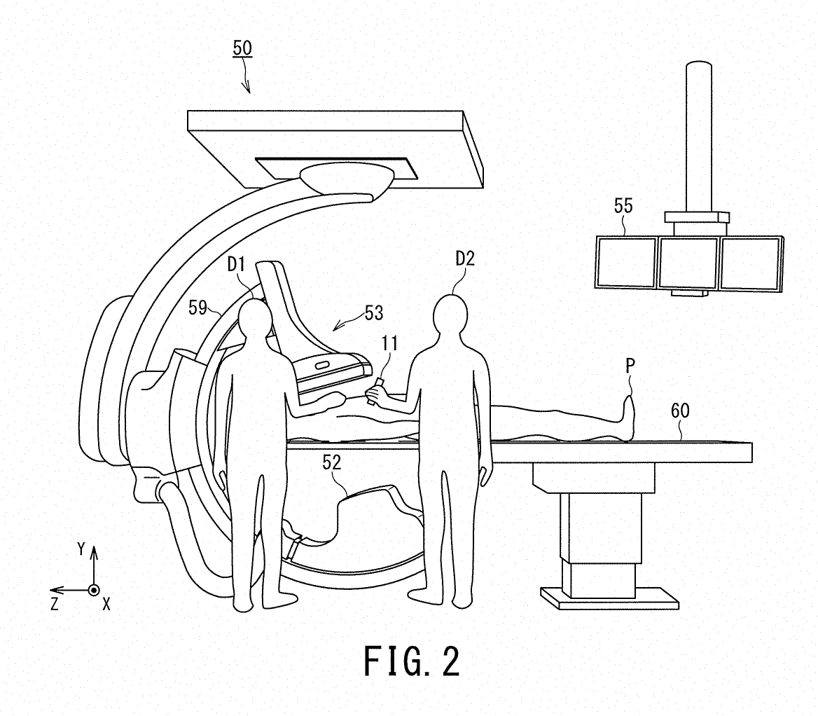

FIG. 1 is a schematic diagram illustrating a configuration of a medical image diagnosis system according to a first embodiment. FIG. 2 is a diagram illustrating an appearance of the medical image diagnosis system according to the first embodiment.

FIGS. 1 and 2 show the medical image diagnosis system 1 according to the first embodiment. The medical image diagnosis system 1 includes an ultrasonic diagnostic apparatus 10 and an X-ray diagnostic apparatus 50 as the medical image diagnosis apparatus according to the first embodiment. For instance, the X-ray diagnostic apparatus 50 is an apparatus configured to visualize cardiovascular system of a patient by using X-rays, i.e., a so-called Angio apparatus. The ultrasonic diagnostic apparatus 10 may be replaced by an MRI (Magnetic Resonance Imaging) apparatus 10′ or may be provided together with the MRI apparatus 10′. In the first embodiment, unless otherwise specifically noted, a description will be given of a case where only the ultrasonic diagnostic apparatus 10 is provided among the ultrasonic diagnostic apparatus 10 and the MRI apparatus 10′.

The ultrasonic diagnostic apparatus 10 includes an ultrasonic probe 11, a main body 12, an input interface 13, a display 14 and a position sensor 15. In some cases, the configuration of main body 12 alone is called an ultrasonic diagnostic apparatus. In some cases, the configuration obtained by adding at least one of the ultrasonic probe 11, the input interface 13, the display 14 and the position sensor 15 to the main body 12 is called an ultrasonic diagnostic apparatus. In the following, a description will be given of a case where the ultrasonic diagnostic apparatus includes all of the ultrasonic probe 11, the main body 12, the input interface 13, the display 14 and the position sensor 15.

The ultrasonic probe 11 includes plural microscopic transducers (i.e., piezoelectric elements) on its front surface, and performs transmission/reception of ultrasonic waves to/from a region including a scan target, e.g., a region including an abdominal aortic aneurysm. Each transducer is an electroacoustic transducer, and has a function of converting electric pulses into ultrasonic pulses at the time of transmission and a function of converting reflected waves into electric signals (i.e., reception signals) at the time of reception. The ultrasonic probe 11 is configured to be small in size and lightweight, and is connected to the main body 12 via a cable (or by wireless communication).

The ultrasonic probe 11 can be classified into various types such as a linear type, a convex type, and a sector type depending on difference in scanning method. In addition, the ultrasonic probe 11 can be classified into a 1D array probe and a 2D array probe depending on array dimensions. In the 1D array probe, plural transducers are one-dimensionally arrayed in the azimuth direction. In the 2D array probe, plural transducers are two-dimensionally arrayed in the azimuth direction and in the elevation direction. The 1D array probe includes a probe in which a small number of transducers are arrayed in the elevation direction.

When a 3D scan, that is, a volume scan is executed, the ultrasonic probe 11 may be configured as a 2D array probe that executes a scan method such as a linear type, a convex type, and a sector type. Additionally or alternatively, when a volume scan is executed, the ultrasonic probe 11 may be configured as a 1D array probe that executes a scan method such as a linear type, a convex type, and a sector type and has a mechanism of mechanically oscillating in the elevation direction. Such a 1D probe is also called a mechanical 4D probe.

The main body 12 includes a transmission/reception (T/R) circuit 31, a B-mode processing circuit 32, a Doppler processing circuit 33, an image generating circuit 34, an image memory 35, a network interface 36, processing circuitry 37, and an internal memory 38. Although the circuits 31 to 34 are configured as, e.g., an application specific integrated circuit (ASIC), embodiments of the present invention are not limited to such an aspect. All or some of the functions of the circuits 31 to 34 may be achieved by causing the processing circuitry 37 to execute the programs.

The T/R circuit 31 has a transmission circuit and a reception circuit (not shown). Under the control of the processing circuitry 37, the T/R circuit 31 controls transmission directivity and reception directivity in transmission and reception of ultrasonic waves. Although a description will be given of the case where the T/R circuit 31 is provided in the main body 12, the T/R circuit 31 may be provided only in the ultrasonic probe 11 or respective two transmission/reception circuits 31 may be provided in the main body 12 and the ultrasonic probe 11.

The transmission circuit includes circuit components such as a pulse generating circuit, a transmission delay circuit, and a pulsar circuit, and supplies a drive signal to the ultrasonic transducers. The pulse generating circuit repeatedly generates a rate pulse for forming a transmission ultrasonic wave at a predetermined rate frequency. It is necessary to set the delay time for each piezoelectric vibrator separately in order to converge the ultrasonic wave generated from the ultrasonic transducers of the ultrasonic probe 11 into a beam shape and thereby determine the transmission directivity, and the transmission delay circuit gives the delay time for each piezoelectric vibrator to each rate pulse generated by the pulse generating circuit. In addition, the pulsar circuit applies a drive pulse to each ultrasonic vibrator at a timing based on the rate pulse. The transmission delay circuit arbitrarily adjusts the transmission direction of the ultrasonic beam transmitted from the piezoelectric vibrator surface by changing the delay time applied to each rate pulse.

The reception circuit includes circuit components such as an amplifier circuit, an analog to digital (A/D) converter, and an adder. The reception circuit receives echo signals received by the respective ultrasonic transducers and then performs various types of processing on the echo signals so as to generate echo data. The amplifier circuit amplifies the echo signals for each channel and performs gain correction processing on the amplified echo signals. The A/D converter performs A/D conversion on the gain-corrected echo signals, and then gives a delay time necessary for determining the reception directivity to the digital data. The adder performs addition processing of the echo signals processed by the A/D converter so as to generate echo data. Since the addition processing is performed by the adder, the reflection component from the direction corresponding to the reception directivity of each echo signal is emphasized.

Under the control of the processing circuitry 37, the B-mode processing circuit 32 receives the echo data from the reception circuit and performs predetermined processing such as logarithmic amplification and envelope detection processing on the echo data so as to generate so-called B-mode data (two-dimensional or three-dimensional data), signal intensity of which is represented by brightness degree or luminance degree.

Under the control of the processing circuitry 37, the Doppler processing circuit 33 performs frequency analysis on the echo data from the reception circuit so as to acquire the speed information, extracts the blood flow and the tissues by the Doppler effect, and generates so-called Doppler data (two-dimensional or three-dimensional data) obtained by extracting moving state information such as average speed, dispersion, and power for multiple points.

Under the control of the processing circuitry 37, the image generating circuit 34 generates an ultrasonic image expressed in a predetermined brightness range as image data on the basis of the echo signals received by the ultrasonic probe 11. For instance, the image generating circuit 34 uses the two-dimensional B-mode data generated by the B-mode processing circuit 32 so as to generate a B-mode image, in which intensity of the reflected wave is expressed in brightness, as an ultrasonic image. In addition, the image generating circuit 34 generates a color Doppler image as an ultrasonic image on the basis of the two-dimensional Doppler data generated by the Doppler processing circuit 33. The color Doppler image includes an average velocity image representing moving state information, a distributed image, a power image, or a combined image of these images.

The image memory 35 includes a two-dimensional memory, and this two-dimensional memory includes memory cells for plural frames such that plural memory cells are arrayed in the respective two axial directions per frame. Under the control of the processing circuitry 37, the two-dimensional memory as the image memory 35 stores ultrasonic images of one frame or plural frames generated by the image generating circuit 34 as two-dimensional image data.

Under the control of the processing circuitry 37, the image generating circuit 34 performs three-dimensional reconstruction on the ultrasonic image arranged in the two-dimensional memory as the image memory 35 such that interpolation processing is applied in the three-dimensional reconstruction as required, and thereby generates an ultrasonic image as volume data in the three-dimensional memory as the image memory 35. As the interpolation processing method, a known technique is used.

The image memory 35 may include a three-dimensional memory that is a memory equipped with plural memory cells in the three axial directions (i.e., X-axis, Y-axis, and Z-axis directions). The three-dimensional memory as the image memory 35 stores the ultrasonic image generated by the image generating circuit 34 as volume data, under the control of the processing circuitry 37.

The network interface 36 implements various information communication protocols according to the form of the network. In accordance with these various protocols, the network interface 36 connects the main body 12 and equipment such as the X-ray diagnostic apparatus 50 installed outside. Electrical connection via an electronic network can be applied to this connection, for instance. The electronic network means a general information communication network using telecommunication technology, and includes a telephone communication network, an optical fiber communication network, a cable communication network, a satellite communication network, Wifi, Bluetooth (Registered Trademark) in addition to a Wireless/wired hospital base local area network (LAN) and the Internet network.

Further, the network interface 36 may implement various protocols for non-contact wireless communication. In this case, the main body 12 can directly transmit and receive data to/from the ultrasonic probe 11 without going through the network.

The processing circuitry 37 means a processor such as a special-purpose or general-purpose central processing unit (CPU), a micro processor unit (MPU) or a graphics processing unit (GPU), or an ASIC and a programmable logic device. As the programmable logic device, it is possible to use a simple programmable logic device (SPLD), a complex programmable logic device (CPLD), or a field programmable gate array (FPGA), for instance.

The processing circuitry 37 may be constituted by a single circuit or a combination of plural independent circuit components. When the processing circuitry 37 is constituted by a combination of plural independent circuit components, the internal memory 38 may be provided individually for each circuit component or a single internal memory 38 may store all the programs corresponding to the functions of the plural circuit components.

The internal memory 38 is composed of, e.g., a hard disk, an optical disk, or a semiconductor memory element such as a random access memory (RAM) and a flash memory. The internal memory 38 may be composed of a portable medium such as a universal serial bus (USB) memory and a digital video disk (DVD). The internal memory 38 stores various processing programs (including an operating system (OS) in addition to application programs) used in the processing circuitry 37 and data necessary for executing the programs. The OS may include a graphical user interface (GUI), in which graphics are frequently used for displaying information on the display 14 to the operator, and by which basic operations can be performed with the input interface 13.

The input interface 13 includes an input device and a circuit for inputting a signal from the input device that can be operated by the ultrasonic technician D2. The input device is realized by a trackball, a switch, a mouse, a keyboard, a touch pad for performing an input operation by touching the scanning surface, a touch screen in which a display screen and a touch pad are integrated, a noncontact input circuit using an optical sensor, and an audio input circuit, for instance. When the input device is operated by the ultrasonic technician D2, the input interface 13 generates an input signal corresponding to the operation and outputs it to the processing circuitry 37.

The display 14 is configured by a general display output device such as a liquid crystal display or an organic light emitting diode (OLED) display. Further, the display 14 includes a graphics processing unit (GPU) and a video RAM (VRAM), for instance. Under the control of the processing circuitry 37, the display 14 displays the ultrasonic image (e.g., live image) requested for display from the processing circuitry 37.

The position sensor 15 time-sequentially detects plural position data items of the ultrasonic probe 11 so as to output the position data items to the main body 12. As the position sensor 15, there are a type of sensor attached to the ultrasonic probe 11 and a type of sensor provided separately from the ultrasonic probe 11. The latter sensor is an optical sensor, images the characteristic points of the ultrasonic probe 11 as the measurement target from plural positions, and detects each position of the ultrasonic probe 11 on the principle of triangulation. Hereinafter, a description will be given of the case where the position sensor 15 is the former sensor.

The position sensor 15 is attached to the ultrasonic probe 11, detects its own position data, and outputs it to the main body 12. The position data of the position sensor 15 can also be regarded as the position data of ultrasonic probe 11. The position data of the ultrasonic probe 11 includes the position and attitude (tilt angle) of the ultrasonic probe 11. For instance, the attitude of the ultrasonic probe 11 can be detected by causing a non-illustrated magnetic field transmitter to sequentially transmit magnetic fields of the three axes and causing the position sensor 15 to sequentially receive the magnetic fields. The position sensor 15 may be a so-called nine-axis sensor that includes at least one of a three-axis gyro sensor configured to detect angular velocities of the respective three axes in three-dimensional space, a three-axis acceleration sensor configured to detect accelerations of the respective three axes in three-dimensional space, and a three-axis geomagnetic sensor configured to detect earth magnetism of each of three-axis in three-dimensional space.

The X-ray diagnostic apparatus 50 includes a high voltage supply 51, an X-ray irradiator 52, an X-ray detector 53, an input interface 54, a display 55, a network interface 56, processing circuitry 57, an internal memory 58, a C-arm 59 (shown only in FIG. 2) and a bed 60 (shown only in FIG. 2).

The high voltage supply 51 supplies high voltage power to the X-ray tube of the X-ray irradiator 52 under the control of the processing circuitry 57.

The X-ray irradiator 52 is provided at one end of the C-arm 59. The X-ray irradiator 52 is provided with an X-ray tube (i.e., X-ray source) and a movable diaphragm. The X-ray tube receives high voltage power from the high voltage supply 51 and generates X-rays according to the conditions of the high voltage power. The movable diaphragm movably supports diaphragm blades at the X-ray irradiation port of the X-ray tube under the control of the processing circuitry 57, and the diaphragm blades are composed of material that shields X-rays. A radiation-quality adjustment filter (not shown) may be provided on the front surface of the X-ray tube for adjusting the radiation-quality of X-rays generated by the X-ray tube.

The X-ray detector 53 is provided at the other end of the C-arm 59 so as to face the X-ray irradiator 52. The X-ray detector 53 can perform operation along a source-image-distance (SID) direction, i.e., can perform back-and-forth motion under the control of the processing circuitry 57. In addition, the X-ray detector 53 can perform a rotation operation along the rotation direction around the SID direction, i.e., rotational motion, under the control of the processing circuitry 57.

The input interface 54 has a configuration equivalent to that of the input interface 13. When the input interface 54 is operated by the operator D (e.g., an operator D1, the ultrasonic technician D2, or an assistant) in the treatment room, the operation signal is transmitted to the processing circuitry 57.

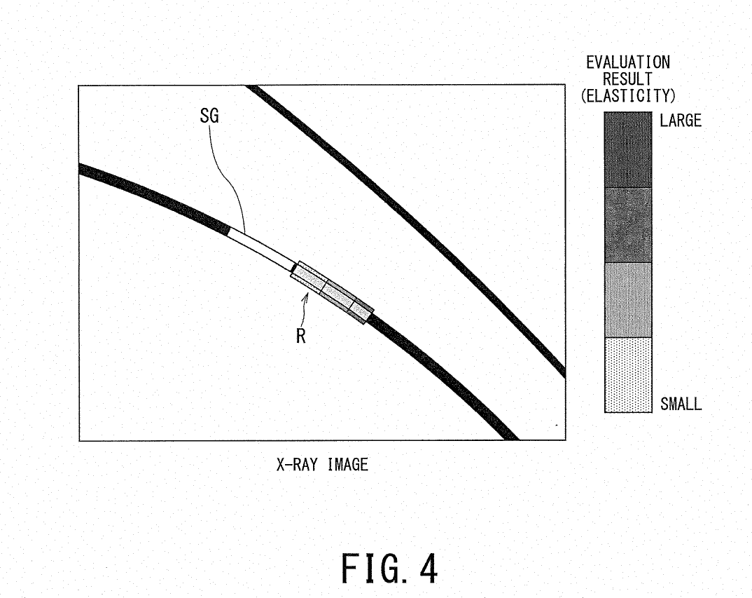

The display 55 has a configuration equivalent to that of the display 14. The display 55 displays the ultrasonic image generated according to ultrasonic imaging and the X-ray image generated according to X-ray imaging. For instance, during interventional operation or treatment, the display 55 displays a superimposed image (e.g., shown in FIG. 4) in which the ultrasonic image is superimposed on the X-ray image, or displays the X-ray image and the ultrasonic image in parallel.

The network interface 56 has a configuration equivalent to that of the network interface 36.

The processing circuitry 57 has a configuration equivalent to that of the processing circuitry 37.

The internal memory 58 has a configuration equivalent to that of the internal memory 38.

The C-arm 59 supports the X-ray irradiator 52 and the X-ray detector 53 such that both face each other. Under the control of processing circuitry 57 or according to manual operation, the C-arm 59 can rotate in the circular arc direction, i.e., the C-arm 59 can rotate in the direction of a cranial view (CRA) and rotate in the direction of a caudal view (CAU). Under the control of processing circuitry 57 or according to manual operation, the C-arm 59 can rotate about its fulcrum, i.e., the C-arm 59 can rotate in the direction of a left anterior oblique view (LAO) and rotate in the direction of a right anterior oblique view (RAO). The C-arm 59 may be configured such that its rotation in the arc direction corresponds to both of the rotation in the LAO direction and the rotation in the RAO direction and its rotation around the fulcrum corresponds to both of the rotation in the CRA direction and the rotation in the CAU direction.

In FIG. 2, the C-arm structure of the X-ray diagnostic apparatus 50 shows a case where the X-ray irradiator 52 is an under-table type positioned below the table of the bed 60. However, embodiments of the present invention are not limited to such an aspect and the X-ray irradiator 52 may be an over-table type positioned above the table. Further, the C-arm 59 may be replaced by an Q arm or the C-arm 59 and the Q arm may be used in combination.

The bed 60 has a table on which an object, e.g., a patient P can be placed. Under the control of the processing circuitry 57, the table can move in the X-axis direction, i.e., can slide in the right-and-left direction. Under the control of the processing circuitry 57, the table can move along the Y-axis direction, i.e., slide in the up-and-down direction. Under the control of the processing circuitry 57, the table can move along the Z axis direction, i.e., slide in the head-and-foot direction. Under the control of the processing circuitry 57, the table can also perform a rolling operation and a tilting operation.

Next, the function of medical image diagnosis system 1 will be described.

The processing circuitry 37 implements an ultrasonic imaging function U1 and an evaluating function U2 by reading out and executing the programs that are stored in the internal memory 38 or are directly incorporated in the processing circuitry 37. Although a description will be given of the case where the functions U1 and U2 are achieved by software, all or a part of the functions U1 and U2 may be achieved by a circuit such as an ASIC provided in the ultrasonic diagnostic apparatus 10.

The ultrasonic imaging function U1 includes a function of causing the respective components to perform ultrasonic imaging for acquiring ultrasonic images by controlling the T/R circuit 31, the B-mode processing circuit 32, the Doppler processing circuit 33, the image generating circuit 34, and the image memory 35. Further, the ultrasonic imaging function U1 includes a function of causing the display 14 to display the ultrasonic images generated according to the ultrasonic imaging.

The evaluating function U2 includes a function of evaluating at least one of elasticity (i.e., hardness or strain) and wall-thickness of the blood vessel in the vicinity of the abdominal (or chest) aortic aneurysm on the basis of the ultrasonic images (e.g., B-mode images) acquired by the ultrasonic imaging function U1. The evaluating function U2 also includes a function of causing the display 14 to display the evaluation result and a function of transmitting the evaluation result to the X-ray diagnostic apparatus 50 via the network interface 36.

The elasticity of the blood vessel can be measured by using an ultrasonic image indicative of hardness distribution of the internal tissues of the patient P, i.e., an image generated by ultrasonic elastography. The ultrasonic elastography is a technique for non-invasively imaging the hardness distribution of tissues such as a blood vessel by using ultrasonic waves, and the following two methods are known as the main methods. One of them is a method of measuring strain distribution at the time of pressurizing the tissues and thereby imaging the relative hardness distribution, and the other of them is a method of measuring propagation velocity distribution of the shear wave at the time of vibrating the tissues and thereby imaging quantitative hardness distribution. Any one of these two method can be used in the present embodiment.

The elasticity of the blood vessel can also be measured by using an MRI image indicative of the hardness distribution of the internal tissues of the patient P, i.e., an image generated by MR elastography. In this case, the MR imaging function (not shown) equivalent to the ultrasonic imaging function U1 and the evaluating function U2 are provided in the MRI apparatus 10′. The MR elastography is a technique to acquire wave propagation as phase information by applying bipolar gradient magnetic fields and applying forced shear vibration in synchronization with the bipolar gradient magnetic fields during magnetic resonance imaging. The evaluating function U2 of the MRI apparatus 10′ performs at least one of the elasticity (hardness or strain) evaluation and the wall thickness evaluation with respect to the blood vessel in the vicinity of the abdominal (or chest) aortic aneurysm, on the basis of the MR image acquired by the MR imaging function.

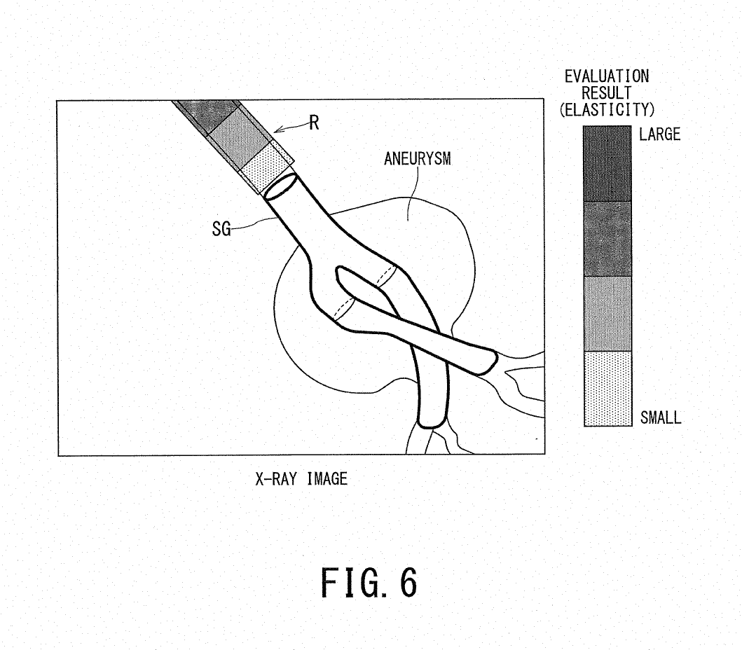

In the present embodiment, the evaluation result means a data set in which values are arranged as brightness values in the blood vessel region in three-dimensional space. The values indicate: the magnitude of elasticity; the magnitude of wall thickness; or both of the magnitude of elasticity and the magnitude of wall thickness. This makes it possible to stepwisely classify the blood vessel region depending on the magnitude of elasticity, the magnitude of wall thickness of the blood vessel, or combination of the magnitude of elasticity and the magnitude of wall thickness of the blood vessel (FIGS. 4 and 6).

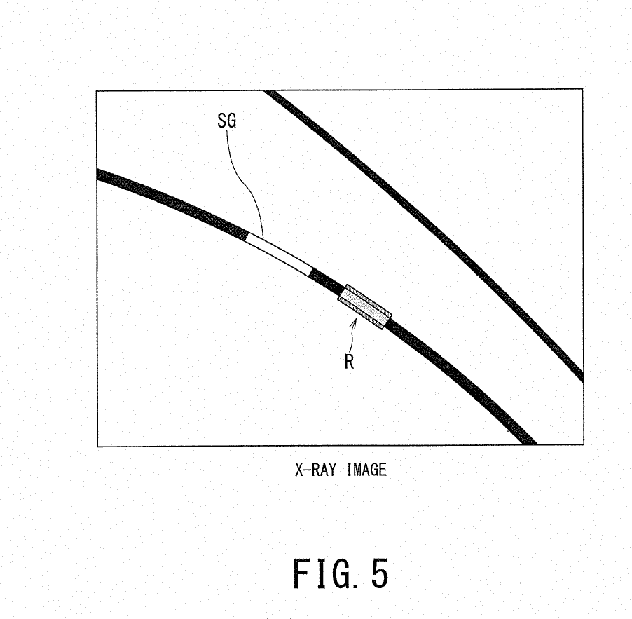

Alternatively, the evaluation result means a data set in which values are arranged as brightness values in the blood vessel region in three-dimensional space. The values indicate: the magnitude of elasticity; the magnitude of wall thickness; or values calculated by binarizing values indicating both of the magnitude of elasticity and the magnitude of wall thickness. In this case, in the blood vessel region of the three-dimensional space, the brightness values are given only to a region in which the elasticity, the wall thickness, or each of the elasticity and the wall thickness is larger (or smaller) than the threshold value. This makes it possible to distinguish, from the blood vessel region, only the portion where the magnitude of elasticity, the magnitude of wall thickness, or both of the magnitude of elasticity and the wall thickness of the blood vessel is larger than a threshold value as shown in FIG. 5.

The evaluation result of at least one of the elasticity evaluation of the blood vessel and the wall thickness evaluation of the blood vessel can be acquired by another method. For instance, the evaluation result may be acquired by using a look-up table (LUT) in which image data including blood vessels and the evaluation results are associated with each other. Additionally, the evaluation result(s) may be acquired by machine learning. When the machine learning is used, the evaluating function U2 calculates feature quantity from the image data including the blood vessels in the vicinity of the abdominal (or chest) aortic aneurysm and outputs the likelihood, which is calculated as the evaluation result by using a matching technique such as a support vector machine (SVM) for matching processing between the feature quantity and the dictionary having learned and registered the past appropriate evaluation result as the correct data (i.e., ground truth) in advance. In addition, the evaluation result may be acquired by deep learning in which a multilayered neural network such as a convolutional neural network (CNN) and a convolutional deep belief network (CDBN) is used as the machine learning.

Depending on the content of interventional operation or treatment, there are at least two cases for where to place the stent graft. In one of the two cases, the stent graft should be placed in the region larger than the threshold value. In the other of the two cases, the stent graft should be placed in the region smaller than the threshold value. Therefore, depending on the content of interventional operation or treatment, either the region larger than the threshold or the region smaller than the threshold is selected.

The processing circuitry 57 implements an X-ray imaging function R, an acquiring function Q1, and a display control function Q2 by reading out and executing the programs that are stored in the internal memory 58 or directly incorporated in the processing circuitry 57. Although a description will be given of the case where the functions R, Q1, and Q2 are achieved by software, all or a part of the functions R, Q1, and Q2 may be achieved by a circuit such as an ASIC provided in the X-ray diagnostic apparatus 50.

The X-ray imaging function R includes a function of controlling the high voltage supply 51, the X-ray irradiator 52, and the X-ray detector 53 and causing them to execute X-ray imaging. In addition, the X-ray imaging function R includes a function of causing the display 55 to display X-ray images generated by X-ray imaging. The X-ray imaging includes X-ray imaging in the fluoroscopic mode and X-ray imaging in the radiographic mode. The radiographic mode means a mode in which relatively strong X-rays are radiated to obtain X-ray images being clearer in contrast, and the fluoroscopic mode is a mode in which relatively weak X-rays are radiated continuously or in a pulsed manner.

The acquiring function Q1 includes a function of acquiring the evaluation result calculated by the evaluating function U2 from the ultrasonic diagnostic apparatus 10. When the MR elastography is adopted, the acquiring function Q1 acquires the evaluation result calculated by the evaluating function U2 of the MRI apparatus 10′ from the MRI apparatus 10′. In addition, the acquiring function Q1 may acquire the evaluation result from an image server for managing the medical image data.

The display control function Q2 includes a function of generating a superimposed image in which the evaluation result acquired by the acquiring function Q1 is superimposed on the X-ray image generated by the X-ray imaging function R, and further includes a function of causing the display 55 to display the superimposed image.

Next, the operation of the medical image diagnosis system 1 will be described. The medical image diagnosis system 1 is applied in the case of placing a stent graft under aortal stent-grafting in the abdomen or chest. In the interventional treatment using the medical image diagnosis system 1, the treatment is performed by using not only the X-ray image obtained from the X-ray diagnostic apparatus 50 but also ultrasonic image obtained from the ultrasonic diagnostic apparatus 10.

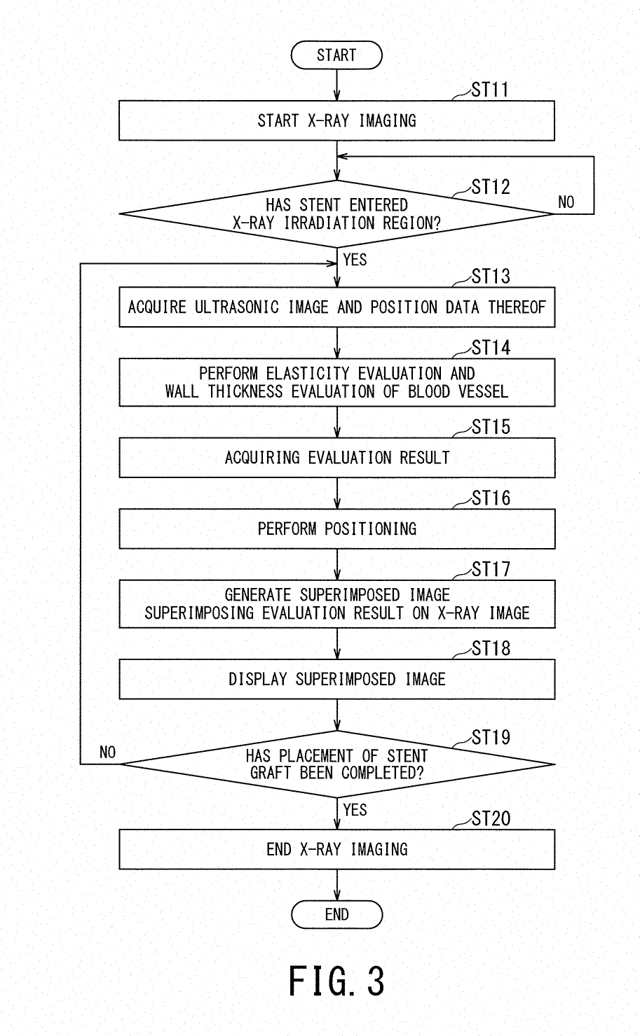

FIG. 3 is a diagram as a flowchart illustrating an operation of the medical image diagnosis system 1. In FIG. 3, each reference sign composed of “ST” and number on the right side indicates the step number of the flowchart.

The operator D1 starts insertion of the catheter having the stent graft attached to its tip into the blood vessel.

The X-ray imaging function R controls the respective components such as the high voltage supply 51, the X-ray irradiator 52, and the X-ray detector 53 so as to start X-ray imaging in the fluoroscopic mode for the patient P (step ST11). The X-ray image generated by X-ray imaging is displayed on the display 55.

The display control function Q2 performs image analysis on the X-ray image of the predetermined frame generated according to the X-ray imaging in the fluoroscopic mode started in step ST11, and determines whether the stent graft has entered the X-ray irradiation region or not (step ST12).

If it is determined as “YES” in step ST12, that is, if it is determined that the stent graft has entered the X-ray irradiation region, the ultrasonic imaging function U1 of the ultrasonic diagnostic apparatus 10 starts ultrasonic imaging (e.g., volume scan) on the vicinity of the stent graft placement position of the patient P to acquire the ultrasonic image (e.g., B-mode image) by controlling the respective components such as the ultrasonic probe 11 and acquires the position data on the position sensor 15 by controlling the position sensor 15 (step ST13). That is, in step ST13, the ultrasonic imaging function U1 acquires the position data on the ultrasonic image obtained from the position data of the position sensor 15.

If it is determined as “NO” in step ST12, that is, if it is determined that the stent graft has not entered the X-ray irradiation region, the display control function Q2 waits until it is determined that the stent graft has entered the X-ray irradiation region.

The evaluating function U2 performs at least one of the elasticity evaluation and the wall thickness evaluation of the blood vessel near the abdominal aortic aneurysm, on the basis of the ultrasonic images acquired in step ST13 (step ST14). The evaluating function U2 freezes a screen, or interrupts updating of the displayed ultrasonic image as necessary to calculate the evaluation result on the basis of the ultrasonic image for which updating is interrupted. The evaluation result means a data set in which values are brightness values arranged in the blood vessel region in three-dimensional space. The values indicate: the magnitude of elasticity; the magnitude of wall thickness; or both of the magnitude of elasticity and the magnitude of wall thickness.

Alternatively, the evaluation result means a data set in which values are arranged as brightness values in the blood vessel region in three-dimensional space. The values indicate: the magnitude of elasticity; the magnitude of wall thickness; or values calculated by binarizing values indicating both of the magnitude of elasticity and the magnitude of wall thickness. In this case, in the blood vessel region of the three-dimensional space, the brightness values are given only to a region in which the elasticity, the wall thickness, or each of the elasticity and the wall thickness is larger (or smaller) than the threshold value.

Incidentally, in each of steps ST13 and ST14, the X-ray imaging started in step ST11 may be temporarily interrupted.

The acquiring function Q1 acquires the evaluation result in step ST14 from the ultrasonic diagnostic apparatus 10 (step ST15). The display control function Q2 performs positioning of the evaluation result on the X-ray image by performing registration between the ultrasonic image and the X-ray image (step ST16).

The display control function Q2 generates a superimposed image by superimposing the evaluation result subjected to the positioning in step ST16 on the X-ray image, which is displayed on the display 55 starting from step ST11 (step ST17).

In steps ST16 and ST17, the display control function Q2 uses the position data on the ultrasonic probe 11 on the side of the ultrasonic diagnostic apparatus 10 and the position data on the C-arm 59 on the side of the X-ray diagnostic apparatus 50 for performing the positioning between both. On the basis of the viewpoint position and the line-of-sight direction specified from the imaging state on the side of the X-ray diagnostic apparatus 50, the display control function Q2 performs rendering of the volume data (e.g., volume rendering or surface rendering) of the evaluation result based on the ultrasonic image so as to generate a projection image of the evaluation result and then superimposes the projection image on the X-ray image.

For instance, the display control function Q2 specifies the focal position of the X-ray irradiator 52 provided at one end of the C-arm 59 as the viewpoint position in the rendering processing of the volume data of the evaluation result based on the ultrasonic image. In addition, the display control function Q2 specifies the imaging direction from the focal position toward the center position of the X-ray detector 53 provided at the other end of the C-arm 59 as the line-of-sight direction in the rendering processing of the volume data of the evaluation result. Thereafter, the display control function Q2 performs the rendering processing on the volume data of the evaluation result on the basis of the specified viewpoint position and line-of-sight direction.

Although the display control function Q2 may project the entire volume data of the evaluation result to generate the projection image and superimpose the projection image on the X-ray image, embodiments of the present invention are not limited to such an aspect. Since the position of the stent graft can be detected from the ultrasonic image, only the area around the stent graft in the volume data of the evaluation result may be projected to generate the projection image and superimpose this projection image on the X-ray image, for instance.

The display control function Q2 causes the display 55 to display the superimposed image generated in step ST17 (step ST18).

FIG. 4 is a first example of a displayed superimposed image. FIG. 5 is a second example of a displayed superimposed image. FIG. 6 is a third example of a displayed superimposed image.

FIG. 4 is a superimposed image in which values indicating the magnitude of elasticity are arranged as brightness values in the blood vessel region of the X-ray image. The superimposed image is generated by assigning a color R to the blood vessel region around the stent graft SG on the X-ray image such that the color R is different depending on the magnitude of elasticity as the evaluation result for each voxel. This makes it possible to stepwisely classify the blood vessel region on the X-ray image displayed on the display 55 depending on the color R reflecting the elasticity, so that a place suitable for placing the stent graft can be presented to the operator D1 by indication of the color R.

FIG. 5 is a superimposed image in which a value obtained by binarizing a value indicative of the magnitude of elasticity is arranged as a brightness value to the blood vessel region of the X-ray image. The superimposed image is generated by assigning a color (e.g., one color R) to the place where the elasticity as the evaluation result is larger than the threshold value among the blood vessel region around the stent graft SG included in the X-ray image. Consequently, the display aspect of using the color R enables the operator D1 to distinguish the place where elasticity is larger than the threshold value, from the blood vessel region on the X-ray image displayed on the display 55. In other words, a place suitable for placing the stent graft can be presented to the operator D1.

FIG. 6 is a superposed image in which values indicating the magnitude of elasticity are arranged as the brightness values to the blood vessel region of the X-ray image. When a non-expandable place (e.g., aneurysm) is present in the blood vessel region as shown in FIG. 6, it is possible to adopt one long stent graft or one combined stent graft composed of plural stent graft components connected to each other such that the adopted stent graft can reach the place beyond the aneurysm, i.e., reach the place where the blood vessel can be expanded. In FIG. 6, the latter stent graft SG is adopted.

The superimposed image is generated by assigning the color R to the blood vessel region around the stent graft SG on the X-ray image in such a manner that the color R for each voxel differs depending on the magnitude of elasticity as the evaluation result. This makes it possible to stepwisely classify the blood vessel region on the X-ray image displayed on the display 55 by the color R in accordance with the magnitude of elasticity, so that the place suitable for placing the stent graft can be presented to the operator D1.

The operator D1 advances the catheter to the predetermined position in the blood vessel while looking at the superposed image shown in FIGS. 4 to 6. Thereafter, the operator D1 inflates the balloon at the predetermined position or expands the expandable spring (stent) so as to expand the blood vessel and then places the stent graft in the blood vessel.

Returning to FIG. 3, the display control function Q2 determines whether the placement of the stent graft has been completed or not (step ST19). For instance, when the operator D1 operates the input interface 54 at the timing of completing the placement of the stent graft, the display control function Q2 can determine that the placement of the stent graft has been completed.

If it is determined as “YES” in step ST19, that is, if it is determined that the placement of the stent graft has been completed, the X-ray imaging function R ends the X-ray imaging started in step ST11 (step ST20). Incidentally, X-ray imaging may be continued during the procedure of retreating the catheter after placing the stent graft.

If it is determined as “NO” in step ST19, that is, if it is determined that the placement of the stent graft has not been completed, the processing returns to step ST13 in which the ultrasonic image and the position data of the position sensor 15 are acquired for the next frame.

According to the medical image diagnosis system 1, on the basis of the elasticity of the blood vessel and the wall thickness of the blood vessel, the place suitable for inflating the balloon or expanding the expandable spring is specified by calculation and the specified place can be presented to the operator D1 via the display 55 of the X-ray diagnostic apparatus 50. Further, according to the medical image diagnosis system 1, the stent graft is placed in an appropriate place at an appropriate pressure by the operator D1 and thus occurrence of endoleak can be reduced.

The endoleak means a phenomenon in which blood flows into the aneurysm. The endoleak is classified into five types, four of which are problems as disease complication. “Type I” is a phenomenon that occurs due to insufficient crimping between the upper or lower sides of the stent graft and the blood vessel wall. “Type II” is a phenomenon caused by back flow of the blood flow in the inferior mesenteric artery and/or the lumbar artery. “Type III” is a phenomenon in which blood leaks from the junction (seam) of the stent graft. “Type IV” is a phenomenon that occurs when blood passes through the stent graft.

### Second Embodiment

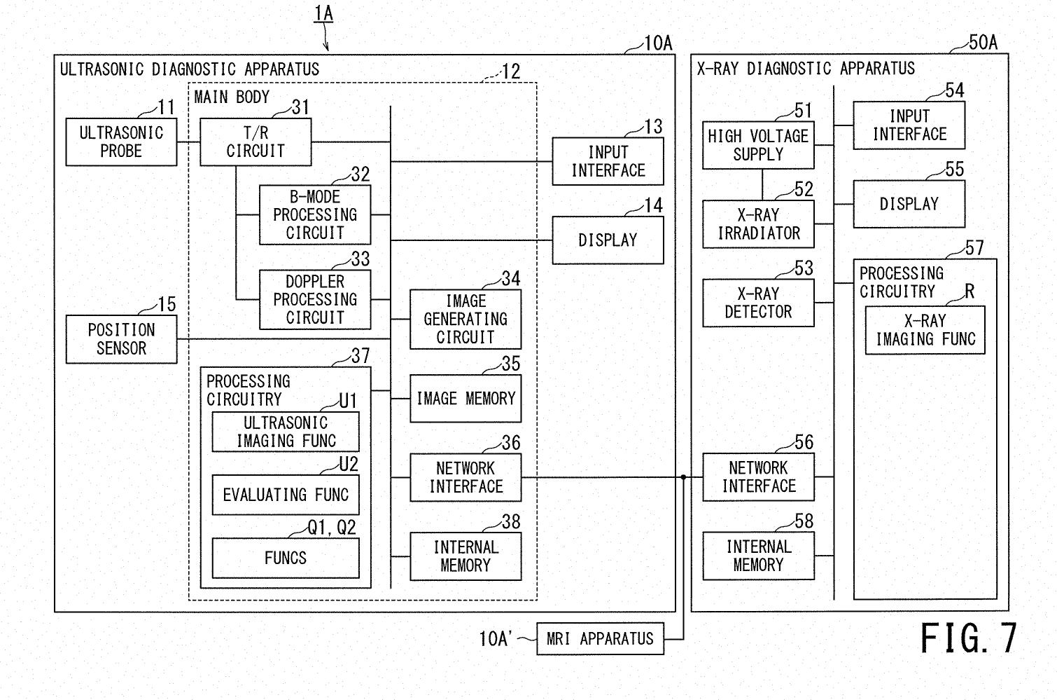

FIG. 7 is a schematic diagram illustrating a configuration of a medical image diagnosis system according to a second embodiment.

FIG. 7 shows a medical image diagnosis system 1A according to the second embodiment. The medical image diagnosis system 1A includes an ultrasonic diagnostic apparatus 10A as the medical image diagnosis apparatus according to the second embodiment and an X-ray diagnostic apparatus 50A. The ultrasonic diagnostic apparatus 10A may be replaced by an MRI apparatus 10A′ or may be provided together with the MRI apparatus 10A′. Unless otherwise specifically noted in the second embodiment, a description will be given of the case where only the ultrasonic diagnostic apparatus 10A is provided among the ultrasonic diagnostic apparatus 10A and the MRI apparatus 10A′.

In FIG. 7, the same components as those in FIG. 1 are denoted by the same reference signs, and duplicate description is omitted.

The processing circuitry 37 of the ultrasonic diagnostic apparatus 10A implements the ultrasonic imaging function U1, the evaluating function U2, the acquiring function Q1, and the display control function Q2 by executing the programs. The processing circuitry 57 of the X-ray diagnostic apparatus 50A implements the X-ray imaging function R by executing the program.

The acquiring function Q1 includes a function of acquiring an X-ray image transmitted from an apparatus installed outside the ultrasonic diagnostic apparatus 10A, e.g., from the X-ray diagnostic apparatus 50A. The functions U1, U2, R, Q1, and Q2 have been described in the first embodiment by referring to FIGS. 1 to 6, and duplicate description is omitted. When MR elastography is performed, the acquiring function Q1 of the MRI apparatus 10A′ acquires an X-ray image transmitted from an apparatus installed outside the MRI apparatus 10A′, e.g., from the X-ray diagnostic apparatus 50A.

According to the medical image diagnosis system 1A, on the basis of the elasticity of the blood vessel and the wall thickness of the blood vessel, a place suitable for inflating the balloon or expanding the expandable spring is specified by calculation and the specified place can be presented to the operator D1 and/or the ultrasonic technician D2 via the display 14 of the ultrasonic diagnostic apparatus 10A. Moreover, according to the medical image diagnosis system 1A, the stent graft is placed at an appropriate place in an appropriate pressure by the operator D1 and thus occurrence of endoleak can be reduced.

(Modification)

So far, a description has been given of the case where the display control function Q2 superimposes the evaluation result calculated by the ultrasonic diagnostic apparatus 10 (or 10A) on the X-ray image so as to generate the superimposed image. However, embodiments of the present invention are not limited to such an aspect. For instance, the display control function Q2 may generate the superimposed image by superimposing information (numerical value or color) indicating pressure difference between the current pressure and the allowable pressure based on the evaluation result on the X-ray image so as to cause the display 55 (or 14) to display the superimposed image.

The allowable pressure means a pressure that allows the balloon to inflate or allows the expandable spring to expand, and is calculated from the evaluation result and the previously registered relationship between the evaluation result and the allowable pressure. The relationship between the evaluation result and the allowable pressure is preset in each medical institution. Additionally, the current pressure means the current pressure value indicated by a device that inflates the balloon or expands the expandable spring.

It should be noted that the information to be superimposed is not limited to the information indicating the pressure difference. For instance, as information to be superimposed, the display control function Q2 may adopt information indicating the ratio of the present pressure to the allowable pressure or may adopt information indicating the ratio of the pressure difference to the allowable pressure.

According to the modification of the medical image diagnosis system 1 (or 1A), during inflation of the balloon or expansion of the spring, the operator D1 can visually recognize how much more pressure can be applied to the blood vessel by checking important parameters such as the pressure difference.

### Third Embodiment

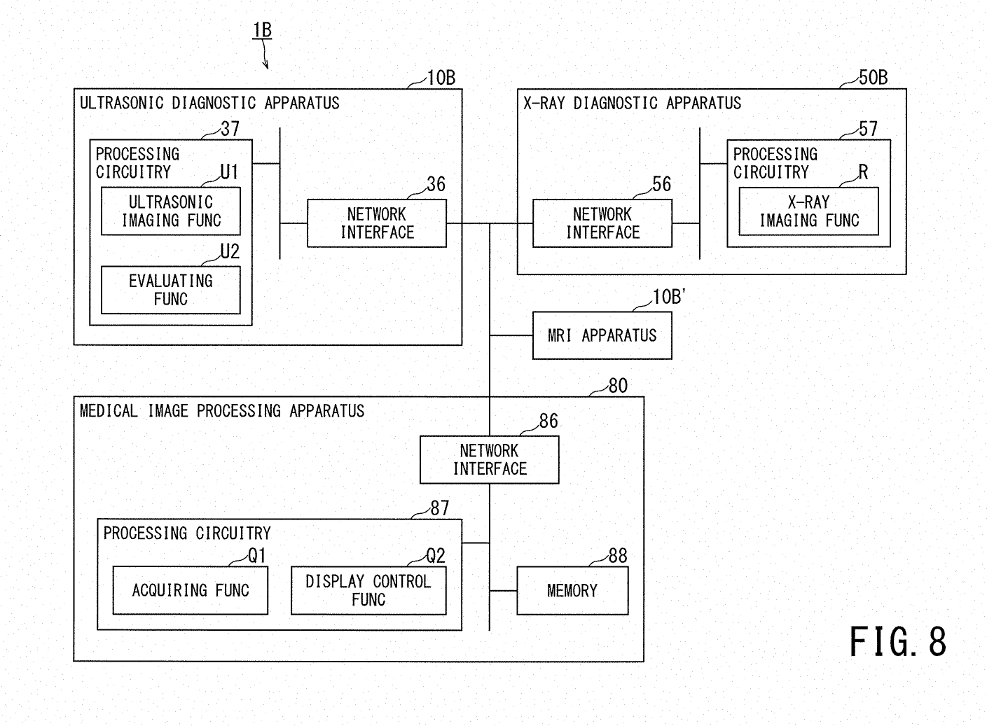

FIG. 8 is a schematic diagram illustrating a configuration of a medical image diagnosis system according to a third embodiment.

FIG. 8 shows a medical image diagnosis system 1B according to the third embodiment. The medical image diagnosis system 1B includes an ultrasonic diagnostic apparatus 10B, an X-ray diagnostic apparatus 50B, and a medical image processing apparatus 80 according to the third embodiment. The medical image processing apparatus 80 is connected to each of the ultrasonic diagnostic apparatus 10B and the X-ray diagnostic apparatus 50B so as to intercommunicate with both. The ultrasonic diagnostic apparatus 10B may be replaced by the MRI apparatus 10B′ or may be provided together with the MRI apparatus 10B′. Unless otherwise specifically noted in the third embodiment, a description will be given of the case where only the ultrasonic diagnostic apparatus 10B is provided among the ultrasonic diagnostic apparatus 10B and the MRI apparatus 10B′.

The medical image processing apparatus 80 is, e.g., a medical image management apparatus, a workstation, or an image interpretation terminal, and is provided on a system connected via a network N.

The medical image processing apparatus 80 may be an off-line apparatus. In this case, the medical image processing apparatus 80 acquires the evaluation result from the ultrasonic diagnostic apparatus 10B via a portable recording medium and acquires the X-ray image from the X-ray diagnostic apparatus 50B via a portable recording medium.

In FIG. 8, the same components as those in FIG. 1 are denoted by the same reference signs, and duplicate description is omitted. Although the ultrasonic diagnostic apparatus 10B includes the ultrasonic probe 11, the main body 12, the input interface 13, the display 14, and the position sensor 15 similarly to the ultrasonic diagnostic apparatuses 10 (shown in FIG. 1) and 10A (shown in FIG. 7), its configuration other than the network interface 36 and processing circuitry 37 is not shown. Likewise, although the X-ray diagnostic apparatus 50B includes the high voltage supply 51, the X-ray irradiator 52, the X-ray detector 53, the input interface 54, the display 55, the network interface 56, the processing circuitry 57, and the internal memory 58 similarly to the X-ray diagnostic apparatuses 50 (shown in FIG. 1) and 50A (shown in FIG. 7), its configuration other than the network interface 56 and processing circuitry 57 is not shown.

The medical image processing apparatus 80 includes a network interface 86, processing circuitry 87, and a memory 88. It should be noted that the medical image processing apparatus 80 may include an input interface having the same configuration as the input interfaces 13 and 54 (shown in FIG. 1), and a display having the same configuration as the displays 14 and 55 (shown in FIG. 1).

The processing circuitry 37 of the ultrasonic diagnostic apparatus 10B implements the ultrasonic imaging function U1 and the evaluating function U2 by executing the programs. The processing circuitry 57 of the X-ray diagnostic apparatus 50B implements the X-ray imaging function R by executing the program.

The processing circuitry 87 of the medical image processing apparatus 80 implements the acquiring function Q1 and the display control function Q2 by reading out and executing the programs, which are stored in the memory 88 or directly incorporated in the processing circuitry 87. Although a description will be given of the case where the functions Q1 and Q2 are achieved by software, all or a part of the functions Q1 and Q2 may be achieved by a circuit such as an ASIC provided in the medical image processing apparatus 80.

The acquiring function Q1 includes a function of acquiring the evaluation result transmitted from the ultrasonic diagnostic apparatus 10B and acquiring the X-ray image transmitted from the X-ray diagnostic apparatus 50B. Since the functions U1, U2, R, Q1, and Q2 have been described in the first embodiment by referring to FIGS. 1 to 6, duplicate description is omitted. When MR elastography is performed, the acquiring function Q1 acquires the evaluation result transmitted from the MRI apparatus 10B′ and acquires the X-ray image transmitted from the X-ray diagnostic apparatus 50B.

According to the medical image diagnosis system 1B, on the basis of the elasticity of the blood vessel and the wall thickness of the blood vessel, a place suitable for inflating the balloon or expanding the expandable spring is specified by calculation and the specified place can be presented to the operator D1 via the display 14 or 55. Furthermore, according to the medical image diagnosis system 1B, the stent graft is placed at an appropriate place in an appropriate pressure by the operator D1 and thus occurrence of endoleak can be reduced.

According to at least one embodiment described above, it is possible to appropriately support the placement of the stent graft by the operator.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. An X-ray diagnostic apparatus comprising: processing circuitry configured to
generate an X-ray image of an object by irradiating the object with X-rays,
acquire an evaluation result of at least one of elasticity evaluation of a blood vessel of the object and thickness evaluation of a wall of the blood vessel of the object,
generate a superimposed image in which the evaluation result is superimposed on the X-ray image, and
display the superimposed image on a display.

2. The X-ray diagnostic apparatus according to claim 1, wherein
the processing circuitry is configured to acquire the evaluation result from an ultrasonic diagnostic apparatus or a magnetic resonance imaging (MRI) apparatus.

3. The X-ray diagnostic apparatus according to claim 1, wherein
the elasticity evaluation and the thickness evaluation are based on an image indicative of hardness distribution of an internal tissue of the object.

4. The X-ray diagnostic apparatus according to claim 1, wherein
the processing circuitry is configured to generate the superimposed image as the evaluation result by superimposing plural colors on the X-ray image, the plural colors depending on at least one of magnitude of elasticity of the blood vessel and magnitude of thickness of the wall of the blood vessel.

5. The X-ray diagnostic apparatus according to claim 1, wherein
the processing circuitry is configured to generate the superimposed image as the evaluation result by superimposing one color on the X-ray image, the one color depending on a value obtained by binarizing at least one of magnitude of elasticity of the blood vessel and magnitude of thickness of the wall of the blood vessel.

6. The X-ray diagnostic apparatus according to claim 1, wherein
the processing circuitry is configured to
generate the superimposed image in which information indicating pressure difference between current pressure and allowable pressure based on the evaluation result, information indicating a ratio of the current pressure to the allowable pressure, or information indicating a ratio of the pressure difference to the allowable pressure is superimposed on the X-ray image, and
display the superimposed image on the display.

7. A medical image processing apparatus comprising:
processing circuitry configured to
acquire an X-ray image of an object and an evaluation result of at least one of elasticity evaluation of a blood vessel of the object and thickness evaluation of a wall of the blood vessel of the object,
generate a superimposed image in which the evaluation result is superimposed on the X-ray image, and
display the superimposed image on a display.

8. A medical image diagnosis apparatus comprising:
processing circuitry configured to calculate an evaluation result of at least one of elasticity evaluation of a blood vessel of an object and thickness evaluation of a wall of the blood vessel of the object,
acquire an X-ray image of the object,
generate a superimposed image in which the evaluation result is superimposed on the X-ray image, and
display the superimposed image on a display.

9. The medical image diagnosis apparatus according to claim 8, wherein
the processing circuitry is configured to
generate the superimposed image in which information indicating pressure difference between current pressure and allowable pressure based on the evaluation result, information indicating a ratio of the current pressure to the allowable pressure, or information indicating a ratio of the pressure difference to the allowable pressure is superimposed on the X-ray image, and
display the superimposed image on the display.
